(19) United States
(12) Patent Application Publication
(10) Pub. No.: US 2003/0096295 A1
(43) Pub. Date: May 22, 2003

(54) Screening methods and therapeutic treatments for pigment dispersion syndrome

(75) Inventors: Michael G. Anderson, Bar Harbor, ME (US); Simon W.M. John, Bar Harbor, ME (US)

Correspondence Address: Kevin M. Farrell, Pierce Atwood, Suite 350, One New Hampshire Avenue, Portsmouth, NH, 03801, US

(73) Assignee: The Jackson Laboratory, Bar Harbor, ME

(21) Appl. No.: 10/338,402

(22) Filed: Jan. 8, 2003

Related U.S. Application Data
(62) Division of application No. 10/005,906, filed on Oct. 26, 2001.

(51) Int. Cl. C12Q 1/68

(52) U.S. Cl. 435/6

(57) ABSTRACT

Disclosed are methods for identifying a genetic disorder associated with pigment dispersion syndrome or pigmentary glaucoma in a mammal. The methods include analytical characterization of a nucleic sample from an afflicted individual, and comparison of this characterization with an otherwise identical characterization of a nucleic acid sample from a non-afflicted individual. Also disclosed are methods for identifying a compound useful in the therapeutic treatment of pigment dispersion syndrome or pigmentary glaucoma. Such methods include the study of melanocyte cell cultures from afflicted and/or non-afflicted individuals, and the effect of test compounds on such cultures. Also disclosed are methods for identifying a therapeutic compound through the use of a mouse model system. Therapeutic methods are also encompassed within the scope of the present disclosure.

### BACKGROUND OF THE INVENTION

Glaucoma accounts for approximately 15% of human blindness worldwide (see, e.g., Thylefors and Negrel, Bull. World Health Organ. 72: 323-326 (1994)) and is the leading cause of blindness in the United States (see, e.g., Leske, Am. J. Epidemiol. 118: 166-191 (1983)). Glaucoma is the eventual outcome of complex disease processes involving the loss of retinal ganglion cells and their associated nerve fibers. This loss produces a characteristic excavation or “cupping” of the optic nerve head. Hallmarks of glaucoma also normally include a characteristic visual field loss.

Some human glaucomas are associated with anterior segment abnormalities such as pigment dispersion syndrome (PDS) and iris atrophy with associated synechiae. See, e.g., Spencer, in: (Glaucoma) Ophthalmic Pathology: An Atlas and Textbook pp. 438-512, Saunders & Co. (1996). The primary causes of these abnormalities are unknown, and their etiology is poorly understood. PDS is relatively common in humans, and was detected in approximately 2.5% and 5.9% of individuals of European and Israeli descent, respectively, that were screened. See, e.g., Ritch, et al., Am. J. Opthalmol. 115: 707-710 (1993). PDS is characterized by slit-like depigmentated iris areas that appear as bright regions when light is reflected through the iris (i.e., iris transillumination defects). Another characteristic of PDS is abnormal accumulation of pigment on structures of the anterior chamber. Studies have shown that up to 50% of individuals diagnosed with PDS develop a form of glaucoma known as pigmentary glaucoma, which is a significant cause of human blindness. See, e.g., Ritcher, et al., Arch. Opthalmol. 104: 211-215 (1986); Migliazzo, et al., Ophthalmology 93: 1528-1536 (1986); Farrar, et al., Am. J. Opthalmol. 108: 223-229 (1989). The initial defect leading to pigment dispersion is, as yet, unknown, and the events contributing to the progression from PDS to pigmentary glaucoma are unclear.

Similarly, in pigmentary glaucoma, abnormally liberated iris pigment and cell debris are liberated into the anterior chamber of the eye and enter the ocular drainage structures, thus disrupting normal aqueous humor drainage and leading to increased IOP and glaucoma. See, e.g., Sugar, Am. J. Ophthalmol. 62: 499-507 (1966); Campbell and Schertzer, in: The Glaucomas, Vol. 2 pp. 975-991, Mosby Publishing (1996).

Accordingly, there remains an, as yet, unfulfilled need for the elucidation of the etiology and clinical consequences of pigmentary glaucoma, for the development of accurate and rapid diagnostic methods, and for the development of efficacious prophylactic and therapeutic treatment modalities.

### SUMMARY OF THE INVENTION

The subject invention relates, in one aspect, to methods for identifying a genetic disorder associated with pigment dispersion syndrome or pigmentary glaucoma in a mammal. The methods include analytical characterization of a nucleic sample from an afflicted individual, and comparison of this characterization with an otherwise identical characterization of a nucleic acid sample from a non-afflicted individual. In another aspect, the present invention relates to methods for identifying a compound useful in the therapeutic treatment of pigment dispersion syndrome or pigmentary glaucoma. Such methods include the study of melanocyte cell cultures from afflicted and/or non-afflicted individuals, and the effect of test compounds on such cultures. The invention also relates to methods for identifying a therapeutic compound through the use of a mouse model system. Therapeutic methods are also encompassed within the scope of the present invention.

### BRIEF DESCRIPTION OF THE DRAWINGS

FIG. 1 is a diagram representing the identification of ipd on mouse chromosome 6.

### DETAILED DESCRIPTION OF THE INVENTION

In one aspect, the present invention relates to a method for identifying a genetic disorder associated with pigment dispersion syndrome or pigmentary glaucoma in a mammal. Initially, a nucleic acid sample from a mammal suspected of being afflicted with pigment dispersion syndrome or pigmentary glaucoma is isolated. The source of the nucleic acid sample need not be tissue-specific, but can be obtained from any cellular source. The specific component of the nucleic acid source which is important in connection with the present invention includes nucleic acid sequences associated with the glycoprotein (transmembrane) nmb gene and the tyrosinase-related protein 1 gene. More specifically, these include cis-acting regulatory sequences (including splice-specifying sequences) and structural sequences which encode amino acids incorporated in the gene product. Examples of nucleic acid preparations of the type described in this paragraph include genomic DNA, mRNA or total RNA preparations.

The nucleic acid sequences associated with the glycoprotein (transmembrane) nmb gene and the tyrosinase-related protein 1 gene, or sequences derived therefrom, are then subjected to analytical characterization. The expression “or sequences derived therefrom” is meant to describe nucleic acid sequences not actually isolated from cells, but rather produced from the nucleic acid sequences isolated from cells in a post-isolation step. Examples of this would include PCR amplification of an isolated sequence, cloning and subsequent amplification of an isolated sequence in living cells, production of cDNA from isolated mRNA, etc.

The analytical characterization to which the nucleic acid sample (or derived sequences) are subjected are capable of resolving single nucleic acid base changes. Such analytical techniques include DNA sequence analysis, techniques specific for the destruction or creation of a restriction enzyme recognition sequence (e.g., restriction fragment length polymorphism analysis), hybridization techniques, gradient denaturation techniques, single-stranded confirmational polymorphism (SSCP) and the like.

Using techniques such as those described above, it is possible to identify single nucleic acid base changes (or larger nucleic acid base changes, such as a deletion) as compared with a sequence obtained from a control population. The control population is preferably genetically (i.e., ethnically) matched. An ideal control population would be ethnically, age, sex and environmentally matched. The use of a control population, rather than merely a control individual, is intended to identify normal allele variation within a population. Normal allele variation, as used herein, refers to naturally occurring variations within the sequence of a given gene among members of a population, the variations not being causative of pigment dispersion syndrome or pigmentary glaucoma.

By comparing the results of the analytical characterization with the nucleic acid from the mammal being tested, with otherwise identical analytical characterization of nucleic acid from a control population, it will be possible to identify base changes in the nucleic acid from the mammal to be tested, which do not appear in the matched control population. Causative mutations will be apparent to one skilled in the art.

Thus, it is possible to associate specific mutant alleles with the disease state. It should be noted that the documentation of therapies followed with any particular individual should be maintained with cross-reference to an implicated mutant allele. This will allow the development of therapies on an allele-by-allele basis.

All methods of the present invention are applicable generally to mammals. Most important, of course, is the identification and treatment of disorders in humans. However, such methods are of importance in other commercially significant contexts involving, for example, livestock and domestic pets. For reference purposes, published sequence information relating to the glycoprotein (transmembrane) nmb gene and the tyrosinase-related protein 1 gene are available. For example, NCBI Accession Nos. include: NT_{—}030006 (human GPNMB genomic sequence); NM_{—}002510 and XM_{—}033627 (human GPNMB mRNA); NT_{—}008484 (human TYRP1 genomic sequence); NM_{—}000550 (human TYRP1 mRNA); AF322054 and AJ251685 (mouse Gpnmb mRNA); NM_{—}031202 (mouse Tyrp1 mRNA).

In another aspect, the present invention relates to methods for the identification of compounds useful in the therapeutic treatment of pigment dispersion syndrome or pigmentary glaucoma. In connection with such methods, cell cultures are initially established from melanocytes isolated from a mammal of interest. The term “melanocytes”, as used herein, is intended to encompass melanin-producing cells. The isolation and establishment of a melanocyte cell culture is routine to one skilled in the art.

The source of the melanocyte is selected following consideration of experimental goals. In one embodiment, cultured melanocytes are contacted with a test compound to be assayed for the ability to decrease melanin production in the melanocytes of the cell culture. Considering the case in which human therapy was the primary interest, one designing the drug screening protocol would have the option of selecting as a source of melanocytes, an individual known to be non-afflicted with pigment dispersion syndrome or pigmentary glaucoma, or an individual known to be afflicted with pigment dispersion syndrome or pigmentary glaucoma.

Arguments can be made to support the choice of either cell source. A prudent experimental design may include both cell sources in parallel experiments. Melanocytes from a non-afflicted individual would be generally considered to be more healthy and vigorous cells. Thus, one would expect such cultures to grow and divide more rapidly, perhaps yielding results in a shorter time frame. Melanocytes from afflicted individuals, on the other hand, more closely correspond to the cell types which are to be the subject of therapeutic intervention. More closely related cells would be preferred by many in the design of such an experiment.

Following the establishment of a melanocyte cell culture (irrespective of the source selected) such cultures are contacted with a test compound to be assayed for the ability to decrease melanin production in the melanocytes of the cell culture. Such a decrease in melanin production can be detected visually, although the design and implementation of an instrument-based detection system is also readily accomplished.

Test compounds to be used in connection with a method of the type described herein are generally small, drug-like compounds. Libraries of such compounds are available commercially. For example, ChemNavigator, Inc., is a San Diego-based cheminformatics company producing a variety of chemical data management products intended to expedite drug discovery. ChemNavigator, Inc., maintains a searchable electronic library of more than two million drug-like compounds. This library can be searched, for example, based on structural similarity to a known compound, to design a custom, information-biased library. The company can further assist in speeding the drug discovery process by procuring the custom library compounds through its international supplier network.

In preferred embodiments of this method of the invention, individual members of the library of drug-like compounds to be tested are introduced into addressed wells in a micro-titer plate. The relevant cell cultures are then incubated in the addressed wells, and the effect of the test compound on pigment production is observed. Compounds which result in decreased melanin production are identified in this manner.

In another aspect, the present invention relates to methods for identifying a compound useful in the therapeutic treatment of pigment dispersion syndrome or pigmentary glaucoma. It is well known that pigment production causes specific cell stresses that are cytotoxic, and deviations caused by these stresses can be monitored in melanocyte cultures. The method includes the determination of certain base line information from a melanocyte culture developed from melanocytes isolated from a mammal known to be afflicted with pigment dispersion syndrome or pigmentary glaucoma. This base line information tends to be information relating to melanosomal-mediated cytotoxicity, mediators of such cytotoxicity and basic survival statistics of the cell culture. These determinations would be made under conditions suitable for growth and division of the melanocytes of the cell culture. Examples of this base line information include melanocyte cell counts over time; radioactive thymidine incorporation; levels of quinones in melanocytes and/or culture medium; levels of oxidative damage to DNA, protein and lipid; cellular morphology indicative of cellular stress involving apoptosis and/or necrosis (e.g., membrane blebbing). Data gathered in this manner will reflect melanocyte stress induced by the increased pigment production.

This base line information is then determined under otherwise identical experimental conditions using a cell culture of melanocytes (i.e., melanin-producing cells) isolated from a mammal known to be non-afflicted with pigment dispersion syndrome or pigmentary glaucoma.

The melanocyte cell culture of step a) is then contacted with a drug-like test compound and maintained under conditions appropriate for cell growth. A test compound of this type would be similar to those discussed above in connection with other embodiments of the present invention. Base line information is again determined and any changes in base line information tending away from that determined in initial studies using melanocytes from an afflicted individual, and tending toward that determined using a melanocyte culture derived from the melanocytes of a non-afflicted individual, indicate that the test compound responsible for the changes in base line data would be a useful therapeutic compound which could then be tested, for example, in a mouse model.

The base line information gathered tends to be information reflective of the general “well-being” of the melanocyte. This can include, for example, cell counts over time; radioactive thymidine incorporation; levels of quinones in melanocytes and/or culture medium; levels of oxidative damage to DNA, protein and lipid; cellular morphology indicative of cellular stress involving apoptosis and/or necrosis. Response to stress can also be included in this base line information. For example, the melanocyte culture can be exposed to a stress-inducing compound such as melanocyte-stimulating hormone (MSH). MSH stimulates melanin production which, over a time course of days, should be self-destructive to wild-type melanocytes in culture. Melanin precursors (e.g., tyrosine or dopa) should enhance cytotoxicity of wild-type melanocyte cells. Expression levels of tyrosinase (the rate-limiting factor in melanin production), Tyrp1 and Gpnmb can also be monitored and recorded as baseline data. The specific examples of base line data provided herein are merely examples, not intended to be limiting.

The invention also relates to the use of a mouse model for identifying a compound useful in the therapeutic treatment of pigment dispersion syndrome or pigmentary glaucoma. In such methods, a mouse model is provided, the mouse model having one or more mutations in either the glycoprotein (transmembrane) nmb gene, or the tyrosinase-related protein 1 gene, or both (the mutations resulting in a form of pigmentary glaucoma). A test compound is then administered to the mouse and an assay for decreased pigment dispersion and decreased initiation and progression of pigmentary glaucoma in the mouse is performed as an indication that the administered test compound is useful in the therapeutic treatment of pigment dispersion syndrome or pigmentary glaucoma.

Test compounds can be administered to the mouse model in any conventional way. This includes, for example, administration directly to the eye as a suspension in an eye drop formulation. Alternatively, the test compound can be administered orally. Any form of injection is also appropriate including, for example, intravenous, subconjunctival, subcutaneous or interperitoneal.

### Exemplification

i) Etiology of IPD and ISA in DBA/2J Mice

In order to elucidate the etiology of pigmentary glaucoma, a murine model, designated DBA/2J (D2), has been genetically-developed. These DBA/2J (D2) mice develop a form of pigmentary glaucoma involving iris pigment dispersion (IPD) and iris stromal atrophy (ISA). See, e.g., John, et al., Invest Opthalmol. Vis. Sci. 39: 951-962 (1998); Chang, et al., Nat. Genet. 21: 405-409 (1999). By use of high-resolution mapping techniques, sequencing, and functional genetic tests, it was demonstrated that IPD and ISA result from mutations in related genes encoding melanosomal proteins. IPD was shown to be caused by a premature stop codon mutation in the glycosylated protein NMB (Gpnmb) gene (designated Gpnmb^{RI50X}), as proven by the occurrence of IPD in only D2 mice that are Gpnmb^{RI50X} homozygotes. D2 mice that are identical in all other respects (except for not being homozygous for Gpnmb^{RI50X}) do not develop IPD. Similarly, ISA was shown to be caused by the recessive tyrosine-related protein 1 mutant allele (designated Tyrp1^{b}), which could be rescued by transgenic introduction of wild-type Tyrp1.

In light of these experimental results, it was hypothesized that IPD and ISA alter melanosomes, allowing toxic intermediates of pigment production to “leak” from melanosomes causing iris disease and subsequent pigmentary glaucoma. This hypothesis is supported by rescue of IPD and ISA in D2 eyes, with substantially decreased pigment production. Similarly, pigment production and mutant melanosomal protein genes are implicated in human pigmentary glaucoma. The finding that hypopigmentation profoundly alleviates D2 disease, indicates that therapeutic strategies which are designed to decrease pigment production, or alleviate the cytotoxic stresses associated with it, will be beneficial in the treatment of human pigmentary glaucoma.

Subsequent to a progressive iris disease, inbred D2 mice develop a form of pigmentary glaucoma involving increased intraocular pressure (IOP), retinal ganglion cell loss, and optic nerve head excavation. See, e.g., John, et al., Invest Opthalmol. Vis. Sci. 39: 951-962 (1998). The D2 iris disease is the consequence of two genetically separable traits (i.e., IPD and ISA) whose combined interactions result in severe iris depigmentation. Observed independently, IPD and ISA are associated with related, yet distinct clinical and histological manifestations. See, e.g., Chang, et al., Nat. Genet. 21: 405-409 (1999). As previously discussed, IPD is characterized by a deterioration of the posterior iris pigment epithelium, slit-like transillumination defects, and pronounced pigment dispersion. The clinical features of IPD clinically-resemble human pigment dispersion syndrome (PDS; OMIN 600510), an apparently common cause of glaucoma in humans whose molecular basis remains unknown. See, e.g., Ritch, et al., Am. J. Opthalmol. 115: 707-710 (1993); Gaton, et al., Harefuah 134: 337-339 (1998); Andersen, et al., Arch. Ophthalmol. 115: 384-388 (1997).

ISA is associated with deterioration of anterior iris stroma resulting in a loss of clinically detectable iris stromal complexity and the accumulation of stromal pigment and cell debris in the ocular draining structures. See, Chang, et al., Nat. Genet. 21: 405-409 (1999). Mice that are homozygous for both ipd (the IPD-causing gene) and isa (the ISA-causing gene) have been shown to have earlier onset, more severe iris disease, and more severe glaucoma, than either single mutant. See, Id. The ipd gene has been previously mapped to mouse Chromosome 6 (position 25.5 cM) and the isa gene to mouse Chromosome 4 (position 34.4 cM). See, Id. In order to identify the causative mutation at these loci, intra-subspecific mapping crosses were performed.

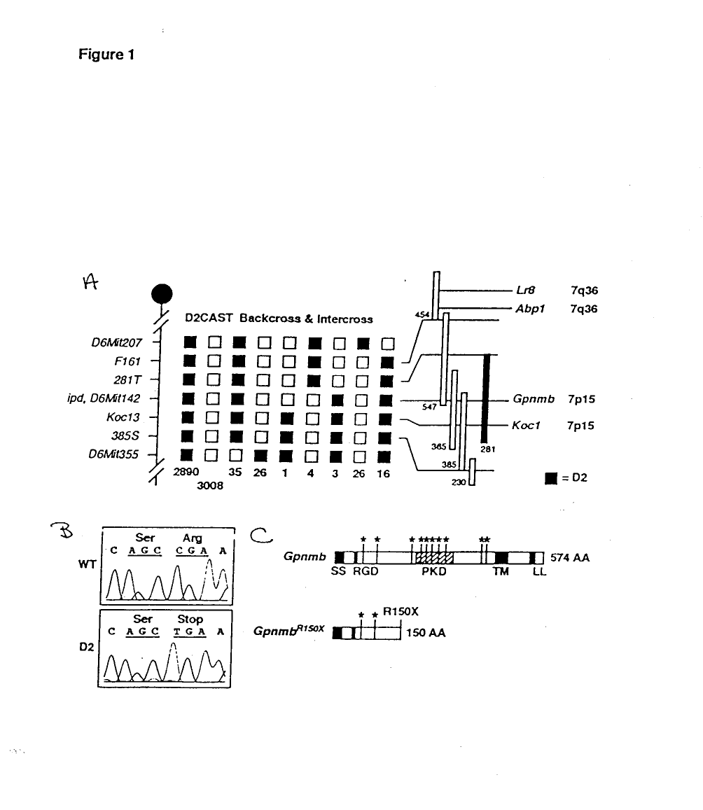

For ipd, a high-resolution genetic map was generated and utilized to identify mice with informative recombinations. See, FIG. 1, Panel A. Aging and clinical analysis of these mice narrowed the ipd-critical region to a 0.13 cM interval, for which a bacterial artificial chromosome (BAC)-derived physical map was generated. Subsequently, additional, new markers (generated from the BAC-derived physical map) further restricted ipd to a single BAC (i.e., 281N17). Analysis of 100 sequences generated from randomly-selected 281N17 fragments identified exons of two genes within this BAC, specifically Koc1 and Gpnmb. Further analysis of a sequenced human contig containing Koc1 and Gpnmb identified two additional putative genes (NLP_{—}1 and PRO2738) potentially within, or flanking the critical region. Of these candidates, only the GPNMB protein is known to be expressed within pigmented cells or the eye. See, e.g., Weterman, et al., Int. J. Cancer 60: 73-81 (1995); Turque, et al., EMBO J. 15: 3338-3350 (1996). Sequencing of the Gpnmb gene from D2 mice detected a premature stop codon mutation, designated Gpnmb^{RI50X}, within the fourth of 11 exons. See, FIG. 1, Panel B. The truncated protein encoded by Gpnmb^{RI50X} is predicted to lack a carboxyl-terminal dileucine melanosomal-sorting motif (see, e.g., Vijayasaradhi, et al., J. Cell. Biol. 130: 807-820 (1995)), as well as the PKD domain that potentially influences protein-protein interactions (see, e.g., Ibraghimov-Beskrovnaya, et al., Hum. Mol. Genet. 9: 1641-1649 (2000)). See, FIG. 1, Panel C.

Interestingly, it was also found that some D2 stocks separated from the modern D2 lineage during the 1970's and 1980's possess the wild-type Gpnmb allele within an otherwise D2 genetic background. For example, sandy (sdy), a recessive coat-color mutation, spontaneously arose on the D2 genetic background in 1983. See, e.g., Swank, et al., Genet. Res. 55: 51-62 (1991). This mutation was maintained at The Jackson Laboratory as a closed breeding colony (DBA/2J-sdy) using sdy/+ obligate heterozygotes, and was subsequently cryopreserved. The DBA/2J-sdy stock was then recovered, retaining its closed breeding status, and it was demonstrated that wild-type and Gpnmb^{RI50X} alleles segregate within this stock.

Similarly, two stocks which were derived from D2 in the 1970's were also shown to possess wild-type Gpnmb alleles. These stocks were DBA/2J-hotfoot (a spontaneous mutation that occurred on the D2 background; see, e.g., Lalouene and Vriz, Genomics 50: 9-13 (1998)) and AKXD-28/Ty (a recombinant inbred line carrying D2 alleles across the chromosomal region flanking Gpnmb; see, e.g., Taylor, in: Genetic Variants and Strains of the Laboratory Mouse, Vol. 2 pp. 1597-1659, Oxford University Press (1996); Anderson, et al., BMC Genet. 2: 1 (2001)). This finding for the DBA/2J-hotfoot and AKXD-28/Ty stocks suggests that Gpnmb^{RI50X} mutation arose during the early 1980's, and subsequently became fixed in the ancestors of the modern D2 mouse strain.

To functionally test whether Gpnmb^{RI50X} is ipd, normally-pigmented DBA/2J-sdy heterozygote mice were aged and analyzed with different Gpnmb genotypes. Because inbred mouse stocks are specifically bred to be genetically identical, these mice are likely identical except for their Gpnmb genotypes. Results showed that only Gpnmb^{RI50X} homozygotes developed IPD (i.e., 6 mice affected/6 mice tested Gpnmb^{RI50X/R150X}; 0 mice affected/13 mice tested Gpnmb^{RI50X/+}; 0 mice affected/4 mice tested Gpnmb^{+/+}—all mice tested were 6-8 months of age). Additionally, the aforementioned colonies of DBA/2J-hotfoot and AKXD-28/Ty mice at The Jackson Laboratory were found to possess wild-type Gpnmb alleles, and did not develop IPD. Accordingly, these results establish that Gpnmb^{RI50X} causes IPD.

For isa, our initial experiments suggested an interesting candidate gene, Tyrp1 (Chang et al., Nat. Genet. 21: 405-409 (1999)). To further evaluate Tyrp1 as a candidate for isa, a total of 1237 meioses were analyzed. In these aged mice, the ISA phenotype and Tyrp1 displayed complete concordance, placing the alleles at the same genetic position (95% confidence interval 0.0 to 0.1 cM) Compared to C57BL/6J mice with normal irides, the Tyrp1 allele of D2 mice (Tyrp1^{b}) encodes a mutant protein containing two amino acid substitutions. Tyrp1^{b} results in a brown instead of black coat color (Taylor, B. A., Recombinant Inbred strains. in Genetic Variants and Strains of the Laboratory Mouse. V. 2 (eds. Lyon, M. F., Rastan, S. & Brown, S. D. M.) 1597-1659 (Oxford University Press, Oxford, 1996)), and other mutant Tyrp1 alleles cause melanocyte death (Shibahara et al., Pigment Cell Res. Suppl.: 90-95 (1992)). To test if ISA is caused by the mutant Tyrp1^{b} allele, a D2 stock of mice transgenic for a BAC containing wild-type Tyrp1 [D2-Tg (Tyrp1), FIG. 3] were generated and analyzed. The BAC has an insert of approximately 88 kb. Analysis of the genomic regions flanking human and mouse Tyrp1 with public and proprietary (Celera) databases indicates that Tyrp1 is likely the only gene in the transgenic insert. Both databases predict no expressed sequences within 61 kb proximal or distal of human TYRP1, indicating that if a similar gene spacing exists in the mouse, the insert would not contain additional genes. This conclusion is also supported by direct analysis of the insert sequence against Celera's largely completed mouse database, indicating that Tyrp1 is the only gene within the rescuing BAC insert. Clinical analysis of aged mice hemizygous for the transgene demonstrates that wild-type Tyrp1 rescues the ISA phenotype, confirming that Tyrp1^{b} causes ISA (FIGS. 3c,d, n=11 mice, aged 6-23 months).

The predicted full-length GPNMB and TYRP1 proteins contain several motifs common to melanosomal proteins, with both proteins sharing similarity to tyrosinase and each other. Based on these similarities, it has been suggested that GPNMB and TYRP1 belong to a common gene family that also includes tyrosinase and silver (Turque et al., Embo. J. 15: 3338-3350 (1996)). BLAST and CLUSTAL analyses both indicate significant similarity between GPNMB and SI, the product of the silver locus in mice implicated as a structural component of the melanosomal matrix (Zdarsky et al., Genetics 126: 443-449 (1990)). TYRP1 is reportedly the most abundant melanosomal glycoprotein (Johnson and Jackson, Nat. Genet. 1: 226-229 (1992)), influences melanosome structure (Kobayashi et al., J. Biol. Chem. 269: 29198-29205 (1994)), and is required for the stabilization of a membrane-bound melanogenic protein complex (Tai et al., Cancer Res. 43: 2773-2779 (1983)). Mouse TYRP1 has various enzymatic activities including catalase (Moyer, F. H., Am. Zool. 6: 43-66 (1966)) and 5,6-dihydroxyindole-2-carboxylic acid (DHICA) oxidase activity (Kobayashi et al., J. Biol. Chem. 273: 31801-31805 (1998)); however, human TYRP1 lacks the latter activity (Halaban and Moellmann, Proc. Natl. Acad. Sci. USA 87: 4809-4813 (1990)). Therefore, structural functions of TYRP1 may reflect a more evolutionarily conserved function.

The aforementioned results led to a hypothesis that the Gpnmb^{RI50X} and Tyrp1^{b} mutations alter melanosomes, allowing pigment production to occur while cytotoxic intermediates of pigment production escape, thus inducing iris disease and subsequent glaucoma. In support of a role for the Tyrp1^{b} mutation in the alteration of melanosomes, it has been previously demonstrated that albino mice with completely abolished pigment production do not exhibit ISA. See, e.g., Chang, et al., Nat. Genet. 21: 405-409 (1999). Thus, to test this hypothesis for both Gpnmb^{RI50X} and Tyrp1^{b}, an epistasis experiment was performed which examined the consequences of reduced pigment production on expression of both IPD and ISA. Importantly, it should be noted that this experiment also tested the potential therapeutic benefit of markedly reduced, but not abolished, pigment production. Pearl (pe), is a coat-color mutation which occurs on the D2 background (DBA/2J-pe). See, e.g., Feng, et al., Hum. Mol. Genet. 8: 323-330 (1999). DBA/2J-pe homozygotes have hypopigmented coats and eyes. Despite homozygosity for Gpnmb^{RI50X} and Tyrp1^{b}, aged homozygous DBA/2J-pe mice do not exhibit either IPD or ISA phenotypes. Additionally, in a similar experiment, hypopigmented eyes from mice homozygous for both sdy and Gpnmb^{RI50X}, also displayed no signs of IPD or ISA. Subsequent histological analysis of a subset of these eyes confirmed these clinical results. Most importantly, these hypopigmentation mutations also prevented the induction of glaucoma by these mutant genes. Taken together, these results demonstrate that the glaucomatous IPD and ISA phenotypes are dependent upon the level of active pigment production occurring within the adult eye, and indicate that treatments to reduce pigment production can be therapeutically-beneficial in pigmentary glaucoma.

While the molecular basis of pigmentary glaucoma in humans is unknown, recent data suggests that pigment production and mutant melanosomal protein genes may similarly contribute to human pigmentary glaucoma. The GPNMB-encoding region among affected individuals of four families segregating for PDS have been sequenced, although no mutations have been detected. Further studies analyzing additional PDS patients and the full GPNMB locus (including the regulatory sequences) are currently being performed. If a similar molecular etiology underlies human pigmentary glaucoma, several possibilities warrant mentioning. First, even though the drug Latanoprost (over 43 million prescriptions have been filled) lowers IOP in the short-term, the induction of melanogenic processes and increased iris pigmentation (see, e.g., Wistrand, et al., Surv. Ophthalmol. 41 (Suppl. 2): S129-138 (1997); Stjemschantz, et al., Acta. Ophthalmol. Scand. 78: 618-620 (2000)) by this commonly-utilized IOP-lowering glaucoma medication may have adverse long-term effects for mammals with pigmentary glaucoma. Second, since UV light induces melanogenic pathways (see, e.g., Nylander, et al., J. Pathol. 90: 39-46 (2000)), exposure to bright sunlight may acutely exacerbate iris pigment dispersion. Third, the results disclosed herein predict that conditions decreasing iris pigmentation should offer protection against pigmentary glaucoma. This study tested this hypothesis by demonstrating that mice with hypopigmented eyes are spared from pigment dispersion and glaucomatous damage. Interestingly, known human TYRP1 mutations have been shown to cause OCA3, a form of oculocutaneous albinism (OMIM 203290), with no reported increased risk of pigmentary glaucoma. See, e.g., Boissy, et al., Am. J. Hum. Genet. 58: 1145-1156 (1996); Manga, et al., Am. J. Hum. Genet. 61: 1095-1101 (1997).

The TYRP1 protein is a member of a multiprotein complex required for stabilization of tyrosinase, the enzyme that catalyzes the first committed step in pigment production. See, e.g., Kobayashi, et al., J. Biol. Chem. 273: 31801-31805 (1998). Because the OCA3 mutations cause oculocutaneous albinism, they may be “self-rescuing” with respect to pigmentary glaucoma, as the mutant TYRP1 protein may not stabilize tyrosinase, thus resulting in greatly decreased production of cytotoxic intermediates of melanogenesis. However, it still remains to be determined if alleles which are permissive for normal levels of iris pigmentation (e.g., Tyrp1^{b}), contribute to human ocular disease. Future studies with D2 mice will help further elucidate the biological functions of Gpnmb and Tyrp1, as well as the link between melanogenesis and pigmentary glaucoma.

iii) Animal Stocks and Husbandry

All experiments were performed in compliance with the ARVO. Statement for use of animals in ophthalmic and vision research. Mice were housed in cages containing white pine bedding and covered with polyester filters. The environment was kept at 21° C. with a 14 hour light and a 10 hour dark cycle. Mice were fed NIH31 (6% fat) chow ad libitum, and their water was acidified to pH 2.8-3.2. All mouse strains utilized in the present invention were routinely screened for select pathogens by The Jackson Laboratory's routine surveillance program (see, http://www.jax.org for the specific pathogens tested). The mouse strains and abbreviations of mice utilized in the present invention included: DBA/2J (D2); C57BL/6J (B6); and CAST/Ei (CAST). Unless specifically stated to the contrary, all references to D2 refer to modern D2 mice. DBA/2J-sdy; DBA/2J-hotfoot (Grid2^{ho-4J}); and DBA/2J-pe (Ap3b1^{pe-8J}) mice were obtained from The Jackson Laboratory Cyropreservation Service. AKXD-28/Ty mice were obtained from Dr. Ben Taylor. The Gpnmb genotype of these mouse stocks was ascertained by assaying for the presence or absence of a unique PvuII site (CAGCTG) created by the Gpnmb^{RI50X} mutation within PCR products amplified from genomic DNA. DBA/2J-Tg (Tyrp1) 280G21Sj (D2-Tg (Tyrp1)) mice were generated by The Jackson Laboratory Microinjection Service using the Tyrp1-containing BAC 280021, initially obtained from a C57BL/6-derived mouse BAC library (Genome Systems). The presence of a wild-type Tyrp1 allele within 280G21 was confirmed by direct sequencing.

iv) Human Subjects

Two affected members from each of four families affected by an autosomal dominant form of pigment dispersion were selected for sequencing the GPNMB gene. The various clinical features of the affected families have been previously reported. See, e.g., Andersen, et al., Arch. Ophthalmol. 115: 384-388 (1997). Genomic DNA purified from a leukocyte pellet was then used to selectively PCR amplify each exon and splice junction of the GPNMB gene. Purified PCR products were subsequently sequenced using nested oligonucleotide primers.

v) Clinical Examinations

The eyes of mice ranging from 2-24 months of age were examined with a slit-lamp biomicroscope (Haag-Streit) and photographed with a 40× objective lens. Phenotypic assessment of iris stromal atrophy, dispersed pigment, and transillumination was performed according to previously-described criteria. See, e.g., John, et al., Invest. Opthalmol. Vis. Sci. 39: 951-962 (1998); Anderson, et al., BMC Genet. 2: 1 (2001).

vi) Locus Mapping

The isa and ipd loci were mapped using similar strategies utilizing mapping mice fixed for the D2 allele of either isa or ipd, and segregating for the other. As a consequence, recombinants were phenotyped for the presence or absence of the phenotype of interest and the severe physiological consequences resulting from the presence of both alleles. The current mapping crosses were derived from inter-subspecific crosses of D2 and CAST. The ipd mapping included 2539 meioses from an N2 or N3 backcross [(D2CASTXD2)N2×D2] or [(D2CASTXD2)N3×D2] and 3470 meioses from an N3F2 intercross [(D2CASTXD2)N3×[(D2CASTXD2)N3]. The mapping of isa was based upon 870 progeny of an N2 backcross [(D2CASTXD2)N2×D2] and 367 previously-reported meioses. See, e.g., Chang, et al., Nat. Genet. 21: 405-409 (1999).

Mice containing informative recombinations were aged and clinically examined at 4-8 week intervals until mice reached 12 months of age. The presence of both alleles was apparent at 5-6 months, and only mice examined at ages exceeding 7 months were used for subsequently narrowing of the critical intervals. All recombinations were utilized for the genetic maps. Mice with recombinations generated from the intercross but homozygous for CAST alleles were progeny tested by re-mating to D2 for phenotypic interpretation. BACs flanking ipd were isolated from a 129Sv-derived mouse CITB library (Research Genetics). It should be noted, however, that the identity given to each BAC correlated to the plate position it was isolated from; and therefore the reported identities may not correlate precisely with those originally assigned by Research Genetics. The designations of the BACs flanking ipd are: 454F16; 547F2; 281N17; 365K21; 385L17; and 230024. The sequence of BAC 454F16 is publicly available. Polymorphic rnicrosatelite markers were purchased from Research Genetics. BAC end-sequences were used to generate additional markers, including polymorphic microsatellites, single nucleotide polymorphisms, and sequence tagged sites (STS). The sequence and reaction parameters of all unique markers are available upon request. For ipd, a total of 8 overlapping BACs spanning the F161 to 385S interval were isolated and examined for the presence of all expected STS markers to ensure use of non-recombinant BACs. Gpnmb RT-PCR products were amplified from D2 and B6 eyes and the complete coding region sequenced. The amino acid positions for GPNMB and TYRP1 are given based upon the predicted precursor proteins.

vii) Histologic Analysis

Enucleated eyes were fixed for plastic sectioning (0.8% parafonnaldehyde and 1.2% glutaraldehyde in 0.08 M phosphate buffer, pH 7.4 or 4% paraformaldehyde in 0.1 M phosphate buffer, pH 7.2) or for paraffin sectioning (3.2% formaldehyde, 0.7 M acetic acid, 61% ethanol) and iris lesions were classified using previously reported criteria. See, e.g., John, et al., Invest. Opthalmol. Vis. Sci. 39: 951-962 (1998); Anderson, et al., BMC Genet. 2: 1 (2001).

1. A method for identifying a genetic disorder associated with pigment dispersion syndrome or pigmentary glaucoma in a mammal, the method comprising:
a) providing a nucleic acid sample from a mammal suspected of being afflicted with pigment dispersion syndrome or pigmentary glaucoma, the nucleic acid sample comprising nucleotides associated with the glycoprotein (transmembrane) nmb gene and the tyrosinase-related protein 1 gene;
b) performing analytical characterization of the nucleic acid sample, or sequences derived from the nucleic acid sample, by analytical techniques capable of resolving single nucleic acid changes; and
c) comparing the analytical characterization of step b), with the results of an otherwise identical analytical characterization carried out on nucleic acid samples from a mammalian population known to be non-afflicted by pigment dispersion syndrome or pigmentary glaucoma, the identification of one or more nucleic acid changes in the nucleic acid of the mammal of step a), as compared with the nucleic acid sequences of the population of step c), being indicative of a genetic disorder associated with pigment dispersion syndrome or pigmentary glaucoma.

2. The method of claim 1 wherein the nucleic acid sample of step a) is genomic DNA, mRNA or total RNA.

3. The method of claim 1 wherein the step b) nucleic acid sample, or sequences derived from the nucleic acid sample, are genomic DNA, cDNA or mRNA.

4. The method of claim 1 wherein the analytical technique of step b) is DNA sequence analysis.

5. The method of claim 1 wherein the analytical technique of step b) comprises the identification of the creation or destruction of a restriction enzyme recognition sequence.

6. The method of claim 5 wherein the analytical technique is restriction fragment length polymorphism analysis.

7. The method of claim 1 wherein the analytical technique of step b) comprises the detection of specific hybridization of an oligonucleotide probe to the nucleic acid or sequences encoded by the nucleic acid, under stringent hybridization conditions.

8. The method of claim 1 wherein the mammal is a human.

9. The method of claim 1 wherein the mammal is a mouse.

10. The method of claim 1 wherein the mammal is a horse.

11. The method of claim 1 wherein the mammal is a canine.

12. A method for identifying a compound useful in the therapeutic treatment of pigment dispersion syndrome or pigmentary glaucoma, the method comprising:
a) establishing a cell culture from melanocytes (i.e., melanin-producing cells) isolated from a mammal;
b) contacting the cell culture of step a) with a test compound to be assayed for the ability to decrease melanin production in the melanocytes of the cell culture;
c) incubating the cell culture of step b) under conditions appropriate for cell growth and division; and
d) identifying a compound having the ability to decrease melanin production in the melanocytes of the cell culture by comparison of the incubated culture of step c) with an otherwise identical culture containing no test compound.

13. The method of claim 12 wherein the mammal of step a) is known to be afflicted with pigment dispersion syndrome or pigmentary glaucoma.

14. The method of claim 12 wherein the method is carried out in parallel with a plurality of individual cell cultures, each individual cell culture being incubated with a unique test compound.

15. The method of claim 14 wherein the plurality of individual cell cultures are contained in the wells of a multi-welled plate.

16. The method of claim 12 wherein the mammal is a human.

17. The method of claim 12 wherein the mammal is a mouse.

18. The method of claim 12 wherein the mammal is a horse.

19. The method of claim 12 wherein the mammal is a canine.

20. A method for identifying a compound useful in the therapeutic treatment of pigment dispersion syndrome or pigmentary glaucoma, the method comprising:
a) providing a cell culture of melanocytes (i.e., melanin-producing cells) isolated from a mammal known to be afflicted with pigment dispersion syndrome or pigmentary glaucoma;
b) determining base line information for the cell culture of step a);
c) providing a cell culture of melanocytes (i.e., melanin-producing cells) isolated from a mammal known to be non-afflicted with pigment dispersion syndrome or pigmentary glaucoma;
d) determining base line information for the cell culture of step c); and
e) contacting the cell culture of step a) with a test compound and identifying any changes in base line information away from that determined in step b) and toward that determined in step d).

21. The method of claim 20 wherein the baseline information is selected from the group consisting of: cell counts over time; radioactive thymidine incorporation; levels of quinones in cells and/or culture medium; levels of oxidative damage to DNA, protein and lipid; cellular morphology indicative of cellular stress involving apoptosis and/or necrosis.

22. A method for identifying a compound useful in the therapeutic treatment of pigment dispersion syndrome or pigmentary glaucoma, the method comprising:
a) providing a mouse model having one or more mutations in the glycoprotein (transmembrane) nmb gene, or the tyrosinase-related protein 1 gene, or both genes, the mutations resulting in a form of pigmentary glaucoma;
b) administering a test compound to the mouse of step a); and
c) assaying for decreased pigment dispersion and decreased initiation and progression of pigmentary glaucoma in the mouse as an indication that the administered test compound is useful in the therapeutic treatment of pigment dispersion syndrome or pigmentary glaucoma.

23. The method of claim 22 wherein the test compound is administered directly to the eye as a suspension in an eye drop formulation.

24. The method of claim 22 wherein the test compound is administered orally.

25. The method of claim 22 wherein the mode of administration of the test compound is administered intravenous, subconjunctival, subcutaneous or interperitoneal.

26. A therapeutic method for treating a mammal afflicted with pigment dispersion syndrome or pigmentary glaucoma, the method comprising administering to the mammal a therapeutically effective amount of a compound which reduces melanin production in the iris.

27. A therapeutic method for treating a mammal afflicted with pigment dispersion syndrome or pigmentary glaucoma, the method comprising administering to the mammal a therapeutically effective amount of a compound identified by the method of claim 20.

28. A therapeutic method for treating a mammal afflicted with pigment dispersion syndrome or pigmentary glaucoma, the method comprising administering to the mammal a therapeutically effective amount of a compound identified by the method of claim 22.

29. An oligonucleotide which hybridizes specifically to a mutant form of the glycoprotein (transmembrane) nmb gene under stringent hybridization conditions, the mutant form of the glycoprotein (transmembrane) nmb gene being linked with pigment dispersion syndrome or pigmentary glaucoma.

30. An oligonucleotide which hybridizes specifically to a mutant form of the tyrosinase-related protein 1 gene under stringent hybridization conditions, the mutant form of the tyrosinase-related protein 1 gene being linked with pigment dispersion syndrome or pigmentary glaucoma.
